(19) United States
(12) Patent Application Publication
(10) Pub. No.: US 2011/0282028 A1
(43) Pub. Date: Nov. 17, 2011

(54) METHOD FOR IDENTIFYING INHIBITORS OF LIPOTEICHOIC ACID SYNTHASE

(75) Inventors: Jeffery Errington, Newcastle (GB); Kathrin Schirner, Boston, MA (US)

(73) Assignee: UNIVERSITY OF NEWCASTLE UPON TYNE, NEWCASTLE UPON TYNE, NC (GB)

(21) Appl. No.: 13/132,818

(22) PCT Filed: Dec. 4, 2009
(86) PCT No.: PCT/GB2009/002824

§ 371 Date: Aug. 1, 2011

(30) Foreign Application Priority Data
Dec. 5, 2008 (GB) 0822276.2

(51) Int. Cl. C12Q 1/25; C07C 233/18; C07H 17/08; C07K 9/00; C07D 493/04; C07D 499/68; C07D 499/44; C07D 499/72; C07H 15/234; C07H 15/26

(52) U.S. Cl. 530/322; 435/29; 536/13.7; 564/213; 536/7.2; 536/53; 549/361; 540/336; 540/314; 540/338

(57) ABSTRACT

The invention provides a method of identifying an inhibitor of LtaS comprising:
(a) providing bacteria which comprise a mutation in the mbl gene or homologue thereof;
(b) culturing the bacteria of (a) in the presence of a test substance under conditions of low magnesium;
(c) monitoring the growth of the bacteria;
   wherein growth or more rapid growth of the bacteria compared to growth in the absence of the test substance is indicative that the test substance is an inhibitor of LtaS.

### RELATED APPLICATIONS

This application is a national phase application claiming benefit of priority under 35 U.S.C. §371 to Patent Convention Treaty (PCT) International Application Serial No: PCT/GB2009/002824, filed Dec. 4, 2009, incorporates by reference and claims the benefit of priority under Great Britain (GB) Provisional Patent Application No. GB 0822276.2, filed Dec. 5, 2008. The aforementioned applications are explicitly incorporated herein by reference in its entirety and for all purposes.

### FIELD OF THE INVENTION

The invention relates to methods or assays to identify agents that can be used as anti-bacterial agents, for example, as antibiotics.

### BACKGROUND TO THE INVENTION

Lipoteichoic acid (LTA) has recently been shown to be essential for Staphylococcus aureus viability. An enzyme responsible for assembly of LTA in S. aureus has also been described. This enzyme has been named lipoteichoic acid synthase, LtaS. See Gründling and Schneewind, 2007, PNAS 104: 8478-8483.

Homologues of LtaS also exist in other bacteria. For example, Bacillus strains express a homolog, previously referred to as yflE. Gründling and Schneewind (supra) demonstrate that the ltaS homolog of Bacillus subtilis can restore LTA synthesis and the growth of ltaS mutant staphylococci. LtaS inhibition can therefore be used as a target to treat human infections caused by S. aureus or other bacterial pathogens. Although Gründling and Schneewind (supra) suggest that LtaS might be a useful target for identification of inhibitors which could be used as antibacterial compounds, no specific assay methods are suggested. The assay used by Gründling and Schneewind to find the ltaS gene would not be readily adaptable for screening of compounds.

Accordingly, there is a need for an assay to identify inhibitors of LtaS.

Mbl is a bacterial actin homolog that is thought to have a role in cell shape determination by positioning the cell wall synthetic machinery. It is also thought to be involved in the control of other functions including cell plurality and chromosome segregation in various organisms. Bacillus subtilis and many other gram positive bacteria have three actin isoforms, one of which is Mbl, which co-localises with two other actin isoforms MreB and MreBH in helical structures that span the length of the cell, close to the inner surface of the cytosplasmic membrane.

Studies carried out with Bacillus subtilis have shown that mutants of the mbl gene are inviable at normal Mg²⁺ levels. Provision of high concentrations of Mg²⁺, for example, 20 mM rescues such bacillus strains. See Carballido-López et al., 2006, Developmental Cell 11, 399-409.

### SUMMARY OF THE INVENTION

The present inventors have identified that transposon mutagenesis can rescue mbl mutants. In particular, Bacillus strains comprising mbl mutations can be subjected to transposon mutagenesis and plated on a low Mg²⁺ medium to identify and select for suppressor mutations which allow growth of the bacteria. Analysis of the transposon mutants identified that inactivation of the ltaS gene causes rescue of mbl mutants such that such strains can grow at low Mg²⁺ conditions. Accordingly, the present inventors describe assays to identify LtaS inhibitors by identifying substances which are able to rescue growth of mbl mutants on low Mg²⁺ medium. These assays are easy and inexpensive cell-based screening methods that allow for screening of a large number of compounds in a straightforward manner.

Thus, in accordance with the first aspects of the present invention, there is provided a method of identifying an inhibitor of LtaS comprising:
(a) providing a gram positive bacteria which comprise a mutation in the mbl gene or homologue thereof;
(b) culturing the bacteria of (a) in the presence of a test substance under conditions of low magnesium;
(c) monitoring the growth of the bacteria;
   wherein growth or more rapid growth of the bacteria compared to growth in the absence of the test substance is indicative that the test substance is an inhibitor of LtaS.

### DESCRIPTION OF THE FIGURES

FIG. 1—B. subtilis Δmbl is Mg²⁺ dependent

A. Plating efficiency after transformation selecting for deletion of mbl with (left) and without (right) addition of 20 mM Mg²⁺. B. Growth curve of B. subtilis wild-type (triangles) and mbl mutant (squares) at 37° C. in PAB medium without (closed symbols) and with (open symbols) addition of 20 mM Mg²⁺. C-E. Morphology (phase-contrast microscopy) of B. subtilis Δmbl grown in PAB (C) or in PAB supplemented with 20 mM Mg²⁺ (D) in comparison to a wild-type strain grown in PAB (E). Scale bar 5 μm.

FIG. 2—Deletion of ltaS suppresses the Mg²⁺ dependency of mbl mutants:

A. Growth of wild-type (168), mbl mutant (2505), ltaS mutant (4283) and suppressed mbl mutant (Δmbl ΔltaS, 4298) on NA plates with (left) or without (right) addition of 20 mM Mg²⁺. B. Growth curves of wild-type (168, ♦), mbl mutant (2505, ▪), ltaS mutant (4283, ▴) and suppressed mbl mutant (Δmbl ΔltaS, 4298, ◯) in PAB medium at 37° C. C. Phase contrast microscopy of wild-type (168), mbl mutant (2505), ltaS mutant (4283) and mbl ltaS double mutant (4298) grown in PAB medium at 37° C. Scale bar 5 μm.

FIG. 3—Effect of metal ion concentration of viability of wild-type and ltaS mutants: A. Growth of wild-type (168) and ltaS mutant (strain 4286) at 37° C. in on NA plates without additives (left), containing 0.5 mM Mg²⁺ (middle) or with addition of 0.05 mM Mn²⁺ (right). B. Growth of ltaS mutant (strain 4283, left) and wild-type (strain 168, right) on minimal medium plates containing 10, 100 and 500 μM Mg²⁺ as indicated.

### DESCRIPTION OF THE SEQUENCES

Table 4 below sets out the sequences of the genes as discussed in more detail below.

SEQ ID NO: 1 and 2 are the nucleotide and amino acid sequences of yflE of Bacillus subtilis.

SEQ ID NO: 3 and 4 are the nucleotide and amino acid sequences of mbl of Bacillus subtilis.

### DETAILED DESCRIPTION OF THE INVENTION

The present invention provides a method for the identification of an inhibitor of LtaS. LtaS is a lipoteichoic acid synthase. LtaS from Staphylococcus aureus has been identified in the prior art, and is described for example in Gründling and Schneewind (supra). Homologues of this gene are also known in other bacterial strains. For example, Bacillus subtilis carries a homolog previously identified as yflE. The sequence for this gene is set out in SEQ ID NO: 1 and 2.

In accordance with the present invention, a bacterial strain of gram positive bacteria, preferably Bacillus, preferably B. subtilis is provided. The bacterial strain is selected or modified to include a functional mutation in the mbl gene of B. subtilis or a homolog thereof of other gram positive bacteria. Mbl is an actin homolog and has been described previously, for example in Abhayawardhane and Stewart, 1995, J. of Bacteriol. 177: 765-773 and Jones et al., Cell 104, 2001, 913-922.

The nucleotide and amino acid sequences for Mbl are set out in Table 4, and labelled as SEQ ID No 3 and 4 respectively. Typically, a homologue of mbl from another bacteria is one having more than about 50%, 55% or 65% identity, preferably at least 70%, at least 80%, at least 90% and particularly preferably at least 95%, at least 97% or at least 99% identity, with the amino acid sequence of SEQ ID NO: 4. Such variants may include allelic variants. The identity of variants of SEQ ID NO: 4 may be measured over a region of at least 200, at least 250, at least 300, at least 330 or more contiguous amino acids of the sequence shown in SEQ ID NO: 4 or more preferably over the full length of SEQ ID NO: 4.

Amino acid identity may be calculated using any suitable algorithm. For example the UWGCG Package provides the BESTFIT program which can be used to calculate homology (for example used on its default settings) (Devereux et al. (1984) Nucleic Acids Research 12, 387-395). The PILEUP and BLAST algorithms can be used to calculate homology or line up sequences (such as identifying equivalent or corresponding sequences (typically on their default settings), for example as described in Altschul S. F. (1993) J Mol Evol 36:290-300; Altschul, S. F. et al. (1990) J Mol Biol 215:403-10.

Software for performing BLAST analyses is publicly available through the National Center for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al., supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff (1992) Proc. Natl. Acad. Sci. USA 89: 10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90: 5873-5787. One measure of similarity provided by the BLAST algorithm is the smallest sum probability (P(N)), which provides an indication of the probability by which a match between two polynucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

The functional mutation can be any mutation that disrupts the function of the mbl gene. Suitable mutations include mutations which disrupt the open reading frame such that a functional Mbl protein cannot be expressed. Alternatively, the mutation may comprise an insertion, for example by transposon mutagenesis to disrupt expression of the gene. In one embodiment, part or all of the mbl gene is deleted. Typically, where the gene is deleted, at least 50% of the mbl gene is deleted, for example, at least 60%, 70%, 80%, 90% or 95%. Smaller deletions can be included, for example, single base deletions to disrupt the open reading frame or smaller deletions, for example at the N-terminus encoding region such that a functional protein is no longer be expressed. Other mutations that can be incorporated are those mutations causing amino acid substitutions at critical sites in the protein, such as those required for binding of ATP. Any mutation in or around the mbl gene that generates a phenotype in which the cells become more dependent on high concentrations of Mg²⁺ in the growth medium can be used.

mbl mutants are dependent upon Mg²⁺ for growth. Thus the mbl mutants useful in accordance with the present invention are unviable or grow poorly under low Mg²⁺ conditions. Supplementation of the culture medium with Mg²⁺ restores cell growth to such bacterial mutants.

The functional mutations in the mbl gene can disrupt the function of the gene such that a functional protein is no longer expressed. Thus, such mutations may affect chromosome segregation or positioning of the cell wall synthetic machinery. Identification of suitable mutants for use in accordance with the present invention can be carried out through a simple analysis of the ability of such mutants to grow under low or normal Mg²⁺. As explained above, mbl mutants are dependent on Mg²⁺ for growth. The assay methods in accordance with the present invention use high levels of Mg²⁺, and thus, a suitable mutant for use in accordance with the present invention is one in which a mutation in the mbl gene leads to a bacteria which is unviable, or which grows poorly under low Mg²⁺ conditions, for example, in which the doubling time of such a mutant under magnesium concentrations of less than 5 mM is typically greater than 12 hours or greater than 24 hours.

In accordance with the assay methods of the present application, the mbl mutant strains are cultured under conditions of low Mg²⁺ in the presence of a test substance. A test substance which acts as an inhibitor of LtaS restores viability of the bacterial strains under such low Mg²⁺ conditions.

Prior to carrying out the assay methods of the present invention, in the presence of a test substance, the mbl mutant strains can be grown under conditions of high or supplemented Mg²⁺, such that the bacteria can grow under these conditions. Typically, for bacterial growth of mbl mutants, Mg²⁺ is present in the range 1 to 100 mM, preferably 3 mM to 50 mM, preferably 5 mM to 30 mM. For example, growth medium can be supplemented with about 20 mM Mg²⁺. For the purpose of an assay, and completion of the test in a convenient period of time, any medium that supports reasonable growth rate of the mbl mutant (e.g. doubling time greater than 120 min at 37° C.) can be used.

Typically, a bacterial culture of an mbl mutant grown under high Mg²⁺ conditions can be diluted and placed into a sample well. Alternatively, such bacteria can be plated on suitable plates with appropriate growth medium such as agar plates, under low Mg²⁺ conditions. References to low Mg²⁺ conditions relate to magnesium concentrations of less than 3 mM, typically less than 1 mM. Typically, bacteria can be cultured in culture medium which has not been supplemented with Mg²⁺. Thus once the mbl mutant bacteria have been diluted or plated out in low Mg²⁺ conditions, their growth will slow or stop.

Low Mg²⁺ conditions can also be identified and defined with respect to bacterial cultures supplemented with 20 mM Mg²⁺. For example, a Mg²⁺ concentration which leads to a growth rate of less than 50%, typically less than 20% or less than 10% of the growth rate of mbl mutants grown in 20 mM medium can be used to identify suitable low Mg²⁺ conditions to conduct the assays in accordance with the present invention.

In order to carry out the assays of the present invention, test substances are added to the mbl mutant bacteria growing under low Mg²⁺ conditions. For example, test substances can be added to the sample wells or spotted on to plates.

In accordance with the assays of the present invention, bacterial growth of the mbl mutants is monitored in the presence of the test substance. Typically, bacteria are grown under usual temperature and time conditions, for example, between 30 and 45° C., typically 37° C. Levels of bacterial growth can be measured at a defined time point, for example, after 2 hours, 4 hours, 6 hours, 8 hours, 12 hours or 24 hours. Alternatively, bacterial growth can be monitored at regular intervals for example every 15 minutes, 30 minutes, hourly, every 2 hours or every 4 hours. Alternatively, bacterial growth can be monitored continuously.

Bacterial growth can be measured by any suitable method. Typically, optical density or a visual assessment of the growth of the bacteria can be carried out. Other suitable methods include use of a dye that generates a colour change during growth (e.g. due to pH change), centrifugation followed by measurement of wet mass, drying followed by measurement of dry mass, chemical determination of a macromolecular component of cells, such as DNA or protein, or counting of cell number microscopically or by an electronic device such as a Coulter counter or flow cytometer, viable cell count by dilution and plating on a suitable growth medium, supplemented with Mg²⁺. Measurement of bacterial growth identifies those mutants whose growth has been rescued despite the low Mg²⁺ conditions. The ability of a test substance to rescue such growth identifies the test substance as an inhibitor of LtaS.

Once an agent has been identified as an inhibitor of LtaS, further studies can be carried out, for example, to assess whether such agent is specific for LtaS. Typically, such test substances can be formulated as pharmaceutical compositions for subsequent administration as antibiotics.

Agents identified in accordance with the present invention can be used as antibiotics against gram positive bacteria, and in particular those which comprise LtaS or a homologue thereof. In a preferred aspect, such agents are useful in the treatment of Staphylococcus aureus infection. Such agents can be used alone or in combination with other antibiotics.

It will be understood that the specific dose level for any particular patient will depend upon a variety of factors including the activity of the specific compound employed, the age, body weight, general health, sex, diet, time of administration, route of administration, rate of excretion, drug combination and the severity of the particular disease undergoing treatment. Optimum dose levels and frequency of dosing will be determined by clinical trial, but an exemplary dosage would be 0.1-1000 mg per day.

The compounds with which the invention is concerned may be prepared for administration by any route consistent with their pharmacokinetic properties. The orally administrable compositions may be in the form of tablets, capsules, powders, granules, lozenges, liquid or gel preparations, such as oral, topical, or sterile parenteral solutions or suspensions. Tablets and capsules for oral administration may be in unit dose presentation form, and may contain conventional excipients such as binding agents, for example syrup, acacia, gelatin, sorbitol, tragacanth, or polyvinyl-pyrrolidone; fillers, for example lactose, sugar, maize-starch, calcium phosphate, sorbitol or glycine; tabletting lubricant, for example magnesium stearate, talc, polyethylene glycol or silica; disintegrants, for example potato starch, or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives such as suspending agents, for example sorbitol, syrup, methyl cellulose, glucose syrup, gelatin hydrogenated edible fats; emulsifying agents, for example lecithin, sorbitan monooleate, or acacia; non-aqueous vehicles (which may include edible oils), for example almond oil, fractionated coconut oil, oily esters such as glycerine, propylene glycol, or ethyl alcohol; preservatives, for example methyl or propyl p-hydroxybenzoate or sorbic acid, and if desired conventional flavouring or colouring agents.

The active ingredient may also be administered parenterally in a sterile medium. Depending on the vehicle and concentration used, the drug can either be suspended or dissolved in the vehicle.

The invention is hereinafter described in more detail with reference to the following Examples.

### Example 1

### Lethal Effects of Mbl Deletion can be Rescued by High Concentrations of Magnesium

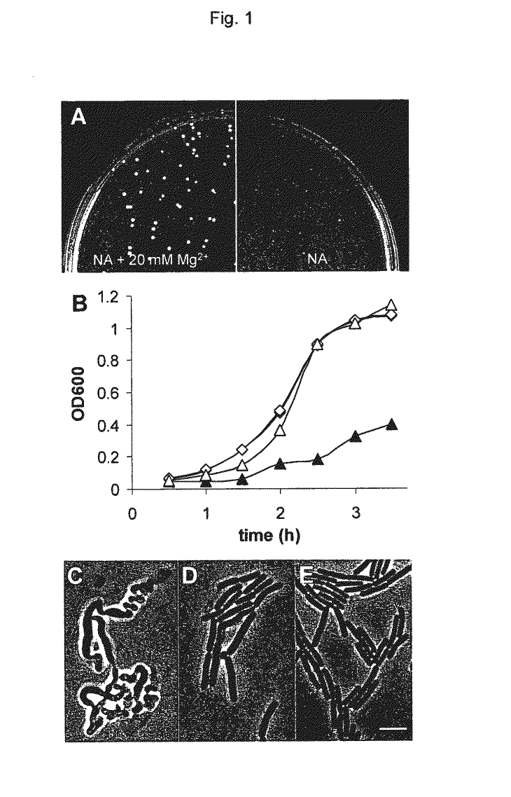

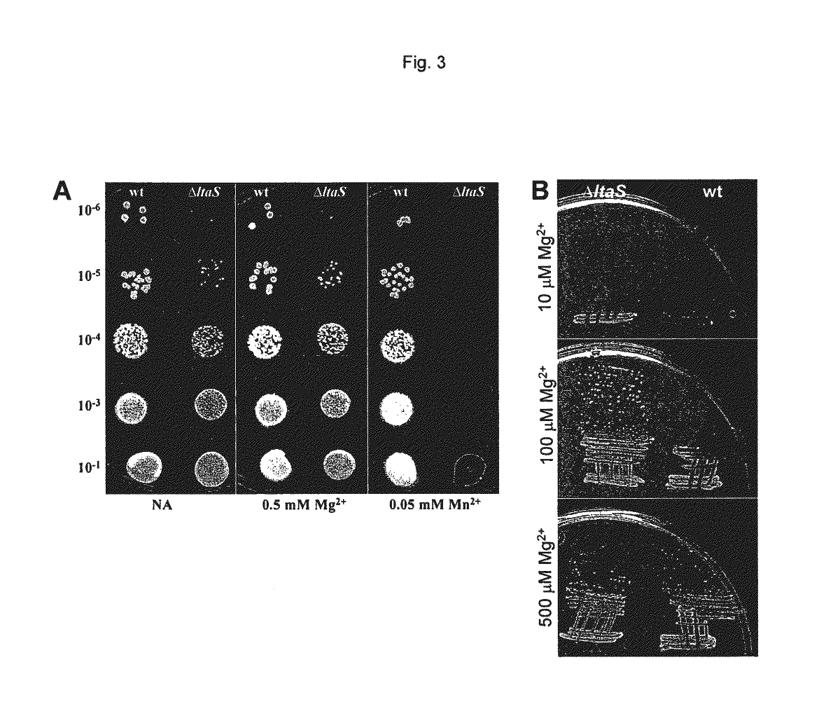

The actin homologue Mbl has been described as non-essential in B. subtilis (Abhayawardhane and Stewart, 1995; Jones et al., 2001), but the former authors had already indicated that mbl mutants are slow growing and tend to pick up mutations that enhance growth. The reported Mg²⁺ dependency of both mreB (Formstone and Errington, 2005) and (though only at low leves) mreBH mutants (Carballido-López et al., 2006) led us to re-construct the mbl deletion strain in the presence of 20 mM Mg²⁺. Selecting for transformants under these conditions resulted in a 10-fold increase in plating efficiency giving relatively small but uniformly shaped colonies (FIG. 1A). Colonies that were picked continued to grow on Nutrient Agar plates supplemented with Mg²⁺, but failed to grow on unsupplemented plates (FIG. 3A). In liquid culture (PAB medium) an elevated magnesium concentration again greatly improved the growth rate (FIG. 1B). Microscopic examination of mutant and wild type cells revealed the characteristic twisted and bloated morphology of the mutant in the unsupplemented medium (FIG. 1C). However, in the presence of Mg²⁺, cell morphology was greatly improved (FIG. 1D). Nevertheless, under high Mg²⁺ conditions the mbl mutant cells still differed from the wild-type in two ways: first, they were slightly bent and irregularly shaped; second, their average diameter was about 12% greater (Table 1). Wild-type cells had their typical straight rod morphology under both conditions (not shown).

Screen for Magnesium Independent Suppressor Mutants of B. subtilis Δmbl

To gain insight into the function of Mbl we screened for mutants in which the Mg²⁺ dependency of the mutant was suppressed. The plasmid pMarB carrying the mariner transposon (Le Breton et al., 2006) was introduced into a freshly constructed Δmbl strain background in the presence of 20 mM Mg²⁺. A library of approximately 60,000 mutants was plated with selection for Mg²⁺ independent growth. Loss of the plasmid pMarB (Erm^{R}), presence of the transposon (Kan^{R}) and disruption of mbl (Spc^{R}) were verified by patching on appropriate antibiotic supplemented plates. Ten strong suppressor strains were chosen and checked for linkage of the transposon insertion to the suppression phenotype by three consecutive back-crosses into the Δmbl mutant background. The sites of transposon insertion were determined by sequencing the products of inverse PCR reactions using primers IPCR1-3 (Le Breton et al., 2006).

In two of the ten suppressor strains, the transposon was found to have independently inserted into the rsgI gene (previously ykrI). RsgI functions as an anti-sigma factor for SigI (Asai et al., 2007). Another hit in the screen was in yflE (three independent insertions), encoding a homologue of the lipoteichoic acid synthase LtaS from S. aureus (Gründling and Schneewind, 2007). Two independent insertions were found in ylxA (synonyms yllC or mraW) which lies in an operon with yllB, ftsL, and pbpB and encodes a protein of unknown function. However, ylxA deletion proved to be not very potent in suppressing the Mg²⁺ dependency of B. subtilis Δmbl (not shown). One transposon insertion each was found in yaaT encoding a protein of unknown function involved in the phosphorelay cascade during initiation of sporulation (Hosoya et al., 2002), in the gene for the glutamate transporter GltT (Slotboom et al., 2001; Tolner et al., 1992), and in pnpA which codes for polynucleotide phosphorylase (Luttinger et al., 1996; Mitra et al., 1996; Wang and Bechhofer, 1996).

Overlapping but Distinct Function of the Actin Homologues in B. subtilis

The finding that mutants of B. subtilis actin homologues MreB and MreBH are sensitive to a low Mg²⁺ concentration (Carballido-López et al., 2006; Formstone and Errington, 2005) led us to re-construct the mbl mutant in the presence of high concentrations of Mg²⁺. The increase in plating efficiency, uniformity of colony shape, and amelioration of the cell morphology recapitulated the earlier findings made for the mreB and mreBH mutants. However, the mutants vary in optimal levels of Mg²⁺: the mreBH mutant requires only about 100-200 μM Mg²⁺ for viability and the cells display a reduced cell width (Carballido-López et al., 2006); the mreB mutant has a higher requirement for Mg²⁺ (2.5 mM), and depletion of the cation results in an increase in cell diameter and ultimately lysis (Formstone and Errington, 2005); finally, the newly constructed mbl mutant requires addition of about 3 mM Mg²⁺ which is in a similar range of the previously described mreB mutant. In unsupplemented medium the strain grows slowly, the cells tend to twist, form chains, swell over their length and are prone to lysis.

In an otherwise wild-type background, the only viable double mutant was Δmbl ΔmreBH, which has a phenotype similar to that of an mbl single mutant. Combinations with ΔmreB were lethal, and depletion of MreB in either mbl or mreBH mutant backgrounds led to a loss of rod-shape and cell death (Defeu Soufo and Graumann, 2006; A. Formstone and J. Errington, unpublished) irrespective of Mg²⁺ levels. Thus, the three MreB-like proteins appear to have overlapping functions, because mreB is essential in strains deleted for any of the other two homologues. However, although the three mutants share certain characteristics like the Mg²⁺ dependency and effects on cell shape, the phenotypic differences between the single mutants show that each has a partially differentiated function.

### Bacterial Strains, Plasmids and Oligonucleotides

B. subtilis strains and plasmids used in this study are listed in Table 2, oligonucleotides in Table 3.

### General Methods

Liquid cultures of B. subtilis strains were grown in Difco Antibiotic Medium 3 (PAB) at 37 or 50° C. as indicated. Nutrient agar (Oxoid) plates were used for growth on solid medium. Minimal concentrations of Mg²⁺ required for growth were determined on Nutrient Agar or Modified Salts Medium (Carballido-López et al., 2006). To all media MgSO₄ was added to the indicated final concentration of Mg²⁺. DNA manipulations and E. coli DH5α transformations were carried out using standard methods (Sambrook, 1989). B. subtilis strains were transformed according to the method of Anagnostopoulos and Spizizen (1961) as modified by Jenkinson (1983). Selection for B. subtilis transformants was carried out on nutrient agar (Oxoid), supplemented with antibiotics, as required, with: kanamycin (5 mg ml⁻¹) chloramphenicol (5 mg ml⁻¹), erythromycin (1 mg ml⁻¹), lincomycin (25 mg ml⁻¹) and/or spectinomycin (50 mg ml⁻¹). IPTG (1 mM) was added as indicated.

### Screen for Mg²⁺—Independent Suppressor Mutants

Random transposon mutagenesis was performed using the mariner based transposon tnYLB-1 as described before (Le Breton et al., 2006). In short, the plasmid pMarB was introduced into an mbl mutant strain (2505) at 30° C. in the presence of high Mg²⁺ concentrations. Individual colonies were picked, grown in LB medium at 37° C. for 8 h, and then plated on nutrient agar plates not supplemented with Mg²⁺ but containing kanamycin to select for the transposon insertions creating Mg²⁺ independent strains. Individual colonies were picked and deletion of mbl (spe^{r}), integration of the transposon tnYLB-1 (neo^{r}) and loss of the plasmid (erm^{s}) were checked by patching on plates containing the appropriate antibiotic. Linkage between transposon insertion and Mg²⁺ independency was verified by back-crossing chromosomal DNA of single colonies three times into an mbl mutant background. Ten strong suppressors were chosen and the site of transposon insertion was determined by inverse PCR amplification and sequencing as described previously (Le Breton et al., 2006).

### Construction of Insertional Deletion Mutants

Chromosomal regions of about 2.5-3 kb flanking the gene(s) to be deleted were PCR amplified using primers mbl-A/mbl-B and mbl-C/mbl-D for the mbl deletion. These PCR products were then ligated to an antibiotic resistance cassette (cat from pCotC; Veening et al., 2006) and then reamplified using the outside primers B+D. Transformation of the resulting PCR products into B. subtilis 168 with selection for the adequate antibiotic then gave rise to strains where the target gene is substituted by an antibiotic resistance cassette. Deletion of the gene and insertion of the resistance cassette was verified by PCR.

### Microscopic Imaging

For microscopy, cells from an overnight liquid or solid culture were diluted into PAB medium supplemented with 20 mM MgSO₄ when required and grown at 37° or 50° C. Cells were mounted on microscope slides covered with a thin film of 1% agarose in minimal medium (Glaser et al., 1997). Staining of the membrane was achieved by mixing 2 μl of Nile Red (Molecular Probe) solution (12.5 mg ml⁻¹) with 600 μl agarose on the slide. Nucleoids were stained by mixing 8 μl of the cell suspension with 2 μl of DAPI (Sigma) solution (1 mg ml⁻¹ in 50% glycerol) in an Eppendorf cup before mounting the sample on the agarose covered slide. Images were aquired with a 14 Sony CoolSnap HQ cooled CCD camera (Roper Scientific) camera attached to a Zeiss Axiovert M135 microscope or to a Zeiss 15 Axiovert 200M microscope. ImageJ (http://rsb.info.nih.gov/ij/) was used to analyse the images, manipulation was limited to altering brightness and contrast to obtain optimal prints.

TABLE 1
Cell dimensions of wild-type and mutant stains

| | Relevant | | | Average |
|---|---|---|---|---|
| Strain | genotype | Temperature | Mg²⁺ added | diameter (±SD) |
| 168 | | 37° C. | | 0.92 (0.07) |
| 168 | | 37° C. | 20 mM | 0.91 (0.07) |
| 168 | | 50° C. | | 0.97 (0.10) |
| 2505 | Δmbl | 37° C. | 20 mM | 1.00 (0.09) |
| 2505 | Δmbl | 50° C. | | 1.12 (0.12) |

Cultures were grown in PAB medium under the conditions indicated.

TABLE 2
Bacterial strains d plasmids

| Strain/plasmid | Relevant genotype | Reference/construction |
|---|---|---|
| B. subtilis | | |
| 168 | trpC2 | laboratory stock |
| 3728 | trpC2 Ωneo3427 ΔmreB | Formstone and Errington, |
| | | 2005 |
| 2505 | trpC2 Ω(mbl::spc) | (Jones et al., 2001) |
| 4261 | trpC2 Δmbl::cat | this work |
| 4283 | trpC2 ΔltaS::neo | this work |
| 4284 | trpC2 ΔltaS:spc | this work |
| 4285 | trpC2 ΔltaS::cat | this work |
| 4286 | trpC2 ΔltaS::erm | this work |
| 4298 | trpC2 Ω(mbl::spc)ΔltaS::neo | this work |
| Plasmids | | |
| PMarB | bla erm P_{ctc}-Himar1 kan | Le Breton et al., 2006 |
| | (TnYLB-1) | |
| pBEST501 | bla neo | Itaya et al., 1989 |
| pVK71 | bla neo::spc | Chary et al., 1997 |
| PMUTIN4 | bla erm Pspac-lacZ lacI | Vagner et al., 1998 |
| pCotC-GFP | bla cat P_{cotC}-cotC-gfp | Veening et al., 2006 |
| pLOSS* | Bla spc Pspac-mcs P div IVA- | Claessen et al., 2008 |
| | lacZ lacI reppLS20 | |

TABLE 3
Oligonucleotides

| Name | Seguence | Description |
|---|---|---|
| IPCR1 | GCTTGTAAATTCTATCATAATTG | IPCR |
| | | amplification |
| IPCR2 | AGGGAATCATTTGAAGGTTGG | IPCR |
| | | amplification |
| IPCR3 | GCATTTAATACTAGCGACGCC | IPCR DNA |
| | | seguencing |
| mbl-A | GCTCACTCTAGACCGAGGTCAATACCAATATCC | XbaI |
| mbl-B | GTGATGAAGCGTCCTATG | |
| mbl-C | CTGAGCGAATTCCGCAAACTAAGCTGATTTCAC | EcoRI |
| mbl-D | CCTATATGGCCTGGAAGAC | |
| mbl-fw | CTCGAGGATCCACCTGGCATTGCCTTCTTG | BamHI |
| mbl-rev | CATACTGAATTCCATGACACCTGTGCCCGATG | EcoRI |
| yflE-A1 | CTAGCAGCATGCGTTCGAGCGAAACGATAG | SphI |
| yflE_A2 | GTACGGTCTAGAGTTCGAGCGAAACGATAG | XbaI |
| yflE-B | CATCGTGATTCCGGCACTC | |
| yflE-C1 | CATCTAGGTACCGAGAGGTTGCCCTCTCC | KpnI |
| yflE_C2 | CTAGCTGAATTCGAGAGGTTGCCCTCTCC | EcoRI |
| yflE-D | CTGCCGTAATGCATGTCAG | |
| yflEfw | GACAGTGGATCCCACTTTCTCCCTCATACG | BamHI |
| yflErev | CATCCAGAATTCGCAGCTGAGGAATTGAGG | EcoRI |

### Example 2

### Deletion of the LTA Synthase YflE Suppresses Mg²⁺ Dependency of Mbl Mutants

We have shown above that mbl mutants are not viable at low [Mg²⁺] and that mutations suppressing this phenotype can be readily obtained. In a collection of transposon induced suppressed mutants were three strains with insertions in the yflE gene. The wild type gene encodes a protein of 649 amino acids with a predicted molecular weight of 74.2 kDa. DNA sequencing showed that each insertion would disrupt the yflE open reading frame, after codons 41, 72 and 387, respectively. While the work was in progress, (Gründling and Schneewind, 2007) showed that a closely related gene (79% identical) in Staphylococcus aureus encodes LTA synthase. They also showed that the yflE gene of B. subtilis could complement the lethal phenotype of ltaS in S. aureus by restoring LTA synthesis. Therefore, hereafter we rename the B. subtilis yflE gene as ltaS.

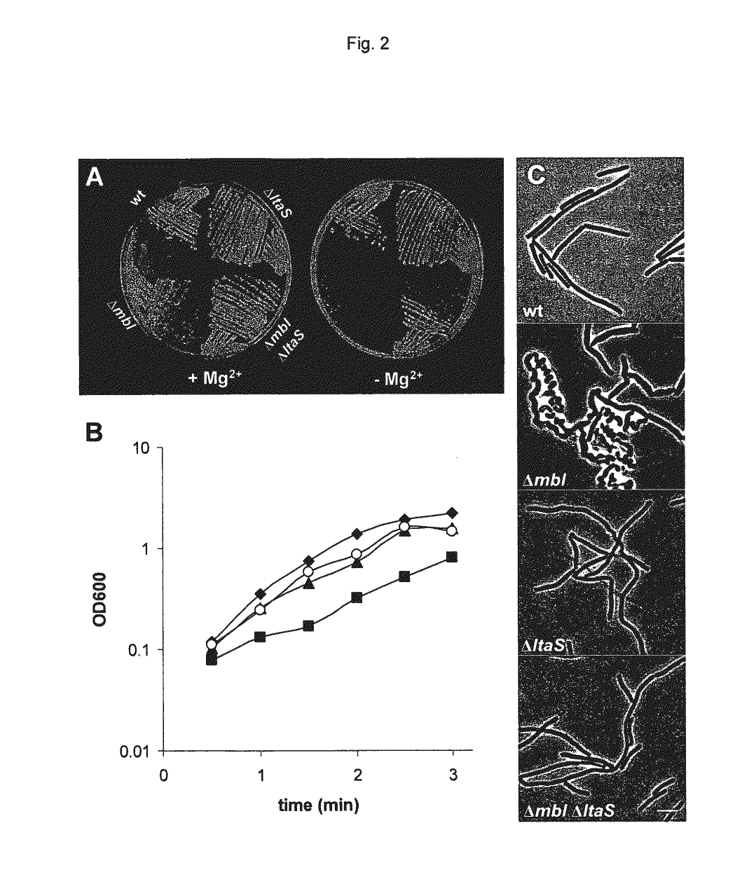

We constructed a complete deletion of the ltaS gene (strains 4283) and confirmed that the ltaS mbl double mutant (strain 4298) is not Mg²⁺ dependent (FIG. 2). Both on plates and in liquid medium (PAB or LB) the double mutant grew without added Mg²⁺ (FIGS. 2A and B), although growth was slower than for the wild type culture. Interestingly, deletion of ltaS also counteracted the typical swelling and twisting of mbl mutant cells; instead the double mutant appeared similar to the ltaS single mutant (FIG. 2C) (see below).

### Effects of Growth Conditions and Metal Ions on LtaS Mutants

The ltaS mutant also exhibited impaired growth depending on the growth medium. To understand the consequences of loss of LTA synthase activity we characterised the growth of the mutant under a range of conditions. The mutant had a slow growth rate in rich media such as PAB (see below) and it failed to grow at all in CH or S media. Systematic analysis of the effects of components of these media added to PAB showed that the mutant strain was particularly sensitive to elevated Mn²⁺ levels. In the examples shown in FIG. 3A, addition of 0.05 mM MnSO₄ to nutrient agar (NA) abolished growth of the mutant, whereas growth of the wild-type was unaffected. Addition of 0.5 mM Mg²⁺ had no effect on growth of the mutant, showing that the effect was not a general sensitivity to divalent cations. On the other hand we noticed that on minimal media plates with defined Mg²⁺ concentrations the ltaS mutant grew at lower Mg²⁺ concentrations than the wild-type strain (FIG. 3B). The lowered requirement for Mg²⁺ might be the reason why a deletion of ltaS suppresses the Mg²⁺ dependent phenotype of mbl and mreB (Formstone and Errington, 2005) mutants. We propose that, consistent with previously suggested functions for LTA in scavenging of Mg²⁺ ions (Neuhaus and Baddiley, 2003), the absence of LTA (synthesized by LtaS) leads to a loss of a buffering zone around the bacterial envelope. As a consequence ions have more immediate access to the cell, leading to a lower requirement for ions with high affinity such as Mg²⁺, which is a co-factor in many bacterial enzymes. At the same time, the toxicity of Mn²⁺ ions increases: these can replace Mg²⁺ because of their similar chemical properties but they do not participate correctly in enzyme function (Cowan, 1995). These results provide direct evidence that LTA has a major role in cell wall physiology and in particular in providing a physicochemical environment that favours the retention of Mg²⁺ over Mn²⁺.

In the process of constructing the deletion strain, we noticed that the ltaS mutant was also hyper-sensitive to various antibiotics and lysozyme. As an example, growth of the ltaS (strain 4285) mutant was abolished at 0.5 μg/ml kanamycin, a concentration that had no discernible effect on the growth of wild-type cells. In other experiments on solid medium the zone of inhibition of all antibiotics tested (kanamycin, ampicillin, vancomycin, penicillin, spectinomycin, erythromycin, lincomycin, carbenicillin) was wider for the ltaS mutant than for the wild-type (not shown). Finally, the mutant also showed increased susceptibility to lysozyme (not shown). The general increase in sensitivity of the mutant to antibiotics and lysozyme is consistent with the notion that LTA also provides a protective layer that restricts the access of many bioactive agents to sensitive sites in the cell envelope.

### Bacterial Strains and Plasmids

B. subtilis strains and plasmids used in this study are listed in Table 2 (supra).

### General Methods

Liquid cultures of B. subtilis strains were grown in Difco Antibiotic Medium 3 (PAB), CH medium (Nicholson & Setlow, 1990), or S-medium (Karamata & Gross, 1970) at 37° C. Nutrient agar (Oxoid) plates were used for growth on solid medium, Modified Salts Medium plates with defined Mg²⁺ concentrations were prepared as described previously (Carballido-López et al., 2006). The given concentration of Mg²⁺ was achieved by addition of MgSO₄ to the medium. DNA manipulations and B. subtilis strains were transformed according to the method of Anagnostopoulos and Spizizen (1961) as modified by Jenkinson (1983). Selection for B. subtilis transformants was carried out on nutrient agar (Oxoid), supplemented with antibiotics, as required, with: kanamycin (5 mg ml⁻¹) chloramphenicol (5 mg ml⁻¹), erythromycin (1 mg ml⁻¹), lincomycin (25 mg ml⁻¹) and/or spectinomycin (50 mg ml⁻¹). To test the sensitivity to cations, cultures were grown to mid-exponential growth phase in PAB medium, then resuspended in PBS to an OD₆₀₀ of 1.0. 10 μl of dilutions 10⁻¹ to 10⁻⁶ in PBS were spotted on NA plates containing MnSO₄ or MgSO₄ in the concentrations as indicated.

### Screen for Mg²⁺ Independent mbl Suppressor Mutants

Random transposon mutagenesis was performed using the mariner based transposon tnYLB-1 as described before (Le Breton et al., 2006). In short, the plasmid pMarB was introduced into an mbl mutant strain (2505) at 30° C. in the presence of high Mg²⁺ concentrations. Individual colonies were picked, grown in LB medium at 37° C. for 8 h, and then plated on nutrient agar plates not supplemented with Mg²⁺ but containing kanamycin to select for the transposon insertions creating Mg²⁺ independent strains. Individual colonies were picked and deletion of mbl (spe^{r}), integration of the transposon tnYLB-1 (neo^{r}) and loss of the plasmid (erm^{s}) were checked by patching on plates containing the appropriate antibiotic. Linkage between transposon insertion and Mg²⁺ independency was verified by back-crossing chromosomal DNA of single colonies three times into an mbl mutant background. Ten strong suppressors were chosen and the site of transposon insertion was determined by inverse PCR amplification and sequencing as described previously (Le Breton et al., 2006).

### Construction of Deletion and Depletion Strains

Genes were deleted by replacing the coding sequence with antibiotic resistance markers. Therefore, approx. 2500 bp up- and downstream of the target genes were amplified using primers yflE-A/yflE-B and yflE-C/yflE-D for the yflE deletion, ligated to the desired resistance cassette and then B. subtilis 168 was transformed with the ligation product, transformants were selected on the appropriate antibiotic and verified by PCR. Resistance cassettes were derived by either restriction or PCR amplification from plasmids [cat from pCotC (Veening et al., 2006); erm from pMUTIN4 (Vagner et al., 1998); neo from pBEST501 (Itaya et al., 1989); spc from pLOSS* (Claessen et al., 2008)].

TABLE 4
Underlined nucleotides in SEQ ID NOS. 1 and 3 indicate the
protein-coding sections of each seguence

| | |
|---|---|
| SEQ ID NO. 1 | |
| attcctttat ttctagaaag atacctt tt ttacatttgg taatatcaaa gcgaaacgtt | 60 |
| gattcgacgg cgtttttcgc cactttctcc ctcatacgat tttcactttt ctaatctgct | 120 |
| gattcgtgtt atattggata cgttcgtttt ttctatcgtt tcgctcgaac tggatcggaa | 180 |
| aaaaggagtg taacaatgaa aacatttata aaagaaagag gactggcctt cttcttaatt | 240 |
| gcggtcgtcc tgttatggat caaaacgtat gtcggttatg tcctgaattt caacttagga | 300 |
| atagacaaca cgatacaaaa aatattgctt tttgtgaatc ctcttagctc aagcttgttc | 360 |
| tttcttggct ttggactctt gttcaagaaa aaattacagc agacagccat tatagtgatt | 420 |
| cattttttaa tgtctttttt actgtacgcc aacattgtgt actacagatt tttcaatgat | 480 |
| tttattacaa ttccggtcat tatgcaggct aaaacaaacg gcggccaact cggtgacagc | 540 |
| gcattttcgc tgatgagacc gactgacgcc ttttacttta tcgatacgat catcctgatc | 600 |
| atcttggcga tcaaagtaaa caagcctgcc gaaacgtcaa gcaaaaaatc gttccgaatt | 660 |
| atttttgcgt cttcaattct tgtgttcttg atcaacctgg cagttgcgga atcagaccgt | 720 |
| cctgaattgc tgacaagatc attcgaccgg aactatcttg tgaaatactt gggaacatac | 780 |
| aatttcacga tttatgacgc tgtacagaat atcaagtcca acagccagcg cgcgcttgcc | 840 |
| gattccagcg acgtaacgga agtagaaaac tacatgaaag ccaattacga tgtgccgaat | 900 |
| aacgtgtatt tcggcaaagc ggaaggaaaa aacgtcattt acgtttcact tgaatctttg | 960 |
| cagtcattta tcatcgacta taaaattgac ggcaaagaag tgacaccatt cttaaataaa | 1020 |
| ctggcacatg ataacgaaac gttctacttt gataactttt tccaccaaac gggacaaggt | 1080 |
| aaaacatctg atgctgaatt tatgatggaa aactctctgt acccgctggc tcaaggttca | 1140 |
| gttttcgtaa acaaagcgca aaacacgctg caatccgttc cggcgattct gaagtctaag | 1200 |
| aattacacat ctgctacttt ccacgggaac acgcagacgt tctggaaccg taacgaaatg | 1260 |
| tacaaggcgg aaggcattga taaattcttt gattctgctt actatgacat gaacgaagaa | 1320 |
| aacacgaaaa actacggcat gaaagacaaa ccgttcttca aagaatcaat gccgctgctg | 1380 |
| gaaagcctgc cgcagccgtt ctatacgaag ttcattaccc tttccaacca cttcccattc | 1440 |
| ggaatggatg agggggatac agacttcccg gctggagact ttggtgactc tgtcgtcgat | 1500 |
| aactatttcc agtcagccca ttaccttgat cagtccattg aacaattctt caatgatctg | 1560 |
| aaaaaagacg ggttatatga taaatcgatt attgtgatgt acggagacca ctacggcatc | 1620 |
| tctgaaaacc acaataaagc gatggcgaaa gtgcttggca aggatgaaat cactgattac | 1680 |
| gacaacgccc agcttcaacg ggtgccgctc tttatccacg ctgccggcgt gaagggcgag | 1740 |
| aaagttcata aatatgccgg agacgttgat gtggctccta ccattctgca tctgctcgga | 1800 |
| gtggatacga aggactatct gatgtccggt tctgatattt tatcgaaaga acaccgtgaa | 1860 |
| gtgattccgt tccgaaacgg agactttatt tcaccgaagt acacgaaaat atccggtaag | 1920 |
| tattacgaca cgaaaaccgg aaaagaactc gatgaatccg aagtcgacaa gtcagaagac | 1980 |
| tcactcgtca agaaggaact tgaaatgtcc gataaaatca taaacggaga cctgctgcgg | 2040 |
| ttctacgagc cgaaaggttt taagaaggtg aatccttctg attatgatta cacaaaacat | 2100 |
| gacgaagatt cttccgaaac gtcaaaggat aacgaagata aataagaaaa agcggagagg | 2160 |
| ttgccctctc cgctttttta tttgacagca gccctcaatt cctcagctgc aaattccaca | 2220 |
| ttcgggccaa taatgacttg aaccgattgc ccgcccgatt tgacaacccc ttttgcgcct | 2280 |
| gctttcttta gcagtgcttc atccaccaaa gcggtatcct tcacagtcag tcgcagcctt | 2340 |
| gt | |
| SEQ ID NO: 2 | |
| MKTFIKERGLAFFLIAVVLLWIKTYVGYVLNFNLGIDNTIQKILLFVNPLSSSLFFLGFG | |
| LLFKKKLQQTAIIVIHFLMSFLLYANIVYYRFFNDFITIPVIMQAKTNGGQLGDSAFSLM | |
| RPTDAFYFIDTIILIILAIKVNKPAETSSKKSFRIIFASSILVFLINLAVAESDRPELLT | |
| RSFDRNYLVKYLGTYNFTIYDAVQNIKSNSQRALADSSDVTEVENYMKANYDVPNNVYFG | |
| KAEGKNVIYVSLESLQSFIIDYKIDGKEVTPFLNKLAHDNETFYFDNFFHQTGQGKTSDA | |
| EFMMENSLYPLAQGSVFVNKAQNTLQSVPAILKSKNYTSATFHGNTQTFWNRNEMYKAEG | |
| IDKFFDSAYYDMNEENTKNYGMKDKPFFKESMPLLESLPQPFYTKFITLSNHFPFGMDEG | |
| DTDFPAGDFGDSVVDNYFQSAHYLDQSIEQFFNDLKKDGLYDKSIIVMYGDHYGISENHN | |
| KAMAKVLGKDEITDYDNAQLQRVPLFIHAAGVKGEKVHKYAGDVDVAPTILHLLGVDTKD | |
| YLMSGSDILSKEHREVIPFRNGDFISPKYTKISGKYYDTKTGKELDESEVDKSEDSLVKK | |
| SEQ ID NO: 3 | |
| aaattctcga aggagagcct gttcagcaat cgtaatcacc tggcattgcc ttcttgaaat | 60 |
| cgttcataaa acatccgcaa aaatttgtaa agaacttatt gtgcttccaa ctttttttct | 120 |
| atattttatg ataatatata taattagggc acaatgtgga tatttactgt gaaacagatt | 180 |
| ttcaaggagg atataaatag atgtttgcaa gggatattgg tattgacctc ggtactgcaa | 240 |
| atgtactgat ccatgttaaa ggtaaaggaa ttgttctgaa tgaaccttcc gttgttgcac | 300 |
| ttgataaaaa cagcggcaaa gtgctggcgg ttggcgaaga ggcaagacga atggttggac | 360 |
| gtacacctgg gaatattgtt gcgattcgcc cgctgaaaga cggagttatt gctgactttg | 420 |
| aagtaacaga agcaatgctg aaacatttta ttaacaagct gaatgtaaaa ggcctgttct | 480 |
| caaagccgcg catgctcatt tgctgcccga cgaatattac atccgttgag caaaaagcaa | 540 |
| ttaaagaagc tgcagaaaaa agcggcggga aacatgtgta ccttgaagaa gaacctaaag | 600 |
| ttgccgctat cggcgcgggt atggaaatat tccagccaag cggtaacatg gttgtagaca | 660 |
| tcggaggcgg gacgacggat atcgcggtta tttcaatggg cgatattgtc acctcctctt | 720 |
| ctattaaaat ggctggggac aagtttgaca tggaaatctt aaattatatc aaacgcgagt | 780 |
| acaagctgct gatcggcgaa cgtactgcgg aggatattaa gattaaagtc gcaactgttt | 840 |
| tcccagacgc acgtcacgag gaaatttcca ttcgcggacg ggacatggtt tccggtcttc | 900 |
| caagaacaat tacagtaaac agtaaagaag ttgaagaagc ccttcgtgaa tctgtcgctg | 960 |
| ttattgttca ggctgcaaaa caagtgctcg aaagaacacc gcctgaactt tctgctgata | 1020 |
| ttattgaccg cggcgttatt attaccggcg gaggcgcgct cttaaacggc cttgaccagc | 1080 |
| tgcttgctga agagctgaag gtaccggtcc tcgttgctga aaatcctatg gattgcgtag | 1140 |
| ccatcggcac aggtgtcatg cttgataata tggacaagct tcctaaacgc aaactaagct | 1200 |
| gatttcacaa acctcattct gaaaaagaat gaggtttttt tatgaaaaag ccttcacgaa | 1260 |
| aagatgttaa atgacgataa taggataaaa tactgagttt ttattataga acgaacgttc | 1320 |
| ctatatgaca actggaaaaa atgccatttt tagaggtggg aaattt tta aaaggattat | 1380 |
| atacagcaac atccgcaat | |
| SEQ ID NO: 4 | |
| MFARDIGIDLGTANVLIHVKGKGIVLNEPSVVALDKNSGKVLAVGEEARRMVGRTPGNIV | |
| AIRPLKDGVIADFEVTEAMLKHFINKLNVKGLFSKPRMLICCPTNITSVEQKAIKEAAEK | |
| SGGKHVYLEEEPKVAAIGAGMEIFQPSGNMVVDIGGGTTDIAVISMGDIVTSSSIKMAGD | |
| KFDMEILNYIKREYKLLIGERTAEDIKIKVATVFPDARHEEISIRGRDMVSGLPRTITVN | |
| SKEVEEALRESVAVIVQAAKQVLERTPPELSADIIDRGVIITGGGALLNGLDQLLAEELK | |
| VPVLVAENPMDCVAIGTGVMLDNMDKLPKRKLS | |

### REFERENCES

Abhayawardhane, Y., and G. C. Stewart. 1995. Bacillus subtilis possesses a second determinant with extensive sequence similarity to the Escherichia coli mreB morphogene. Journal of Bacteriology 177:765-773.
Anagnostopoulos, C., and J. Spizizen. 1961. Requirements for transformation in Bacillus subtilis. J. Bacteriol. 81:741-746.
Asai, K., T. Ootsuji, K. Obata, T. Matsumoto, Y. Fujita, and Y. Sadaie. 2007. Regulatory role of RsgI in sigI expression in Bacillus subtilis. Microbiology 153:92-101.
Carballido-López, R., A. Formstone, Y. Li, S. D. Ehrlich, P. Noirot, and J. Errington. 2006. Actin homolog MreBH governs cell morphogenesis by localization of the cell wall hydrolase LytE. Developmental Cell 11:399-409.
Chary, V. K., E. I. Amaya, and P. J. Piggot. 1997. Neomycin- and spectinomycin-resistance replacement vectors for Bacillus subtilis. FEMS Microbiology Letters 153:135-139.
Claessen, D. R. Emmins, L. W. Hamoen, R. A. Daniel, J. Errington, and D. D. Edwards. 2008. Control of the cell elongation-division cycle by shuttling of PBP1 protein in Bacillus subtilis. Molecular Microbiology 68:1029-1046.
Defeu Soufo, H. J., and P. L. Graumann. 2006. Dynamic localization and interaction with other Bacillus subtilis actin-like proteins are important for the function of MreB. Molecular Microbiology 62: 1340-1356.
Formstone, A., and J. Errington. 2005. A magnesium-dependent mreB null mutant: implications for the role of mreB in Bacillus subtilis. Molecular Microbiology 55:1646-1657.
Glaser, P., M. E. Sharpe, B. Raether, M. Perego, K. Ohlsen, and J. Errington. 1997. Dynamic, mitotic-like behavior of a bacterial protein required for accurate chromosome partitioning. Genes Dev. 11:1160-1168.
Gründling, A., and O, Schneewind. 2007. Synthesis of glycerol phosphate lipoteichoic acid in Staphylococcus aureus. Proceedings of the National Academy of Sciences of the United States of America 104:8478-8483.
Hoper, D., U. Volker, and M. Hecker. 2005. Comprehensive characterization of the contribution of individual SigB-dependent general stress genes to stress resistance of Bacillus subtilis. Journal of Bacteriology 187:2810-2826.
Hosoya, S., K. Asai, N. Ogasawara, M. Takeuchi, and T. Sato. 2002. Mutation in yaaT leads to significant inhibition of phosphorelay during sporulation in Bacillus subtilis. Journal of Bacteriology 184:5545-5553.
Itaya, M., K. Kondo, and T. Tanaka. 1989. A neomycin resistance gene cassette selectable in a single copy state in the Bacillus subtilis chromosome. Nucleic Acids Research 17:4410.
Jenkinson, H. F. 1983 Altered arrangement of proteins in the spore coat of a germination mutant of Bacillus subtilis. Journal of General Microbiology 129:1945-1958.
Jones, L. J., R. Carballido-López, and J. Errington. 2001. Control of cell shape in bacteria: helical, actin-like filaments in Bacillus subtilis. Cell 104:913-922.
Le Breton, Y., N. P. Mohapatra, and W. G. Haldenwang. 2006. In Vivo Random Mutagenesis of Bacillus subtilis by Use of TnYLB-1, a mariner-Based Transposon. Appl. Environ. Microbiol. 72:327-333.
Luttinger, A., J. Hahn, and D. Dubnau. 1996. Polynucleotide phosphorylase is necessary for competence development in Bacillus subtilis. Molecular Microbiology 19:343-356.
Mitra, S., K. Hue, and D. H. Bechhofer. 1996. In vitro processing activity of Bacillus subtilis polynucleotide phosphorylase. Molecular Microbiology 19:329-342.
Sambrook, J., E. F. Fritsch, T. Maniatis. 1989. Molecular Cloning: A Laboratory Manual. Cold Spring Harbor, N.Y.: Cold Spring Harbor Laboratory Press.
Slotboom, D. J., W. N. Konings, and J. S. Lolkema. 2001. Cysteine-scanning mutagenesis reveals a highly amphipathic, pore-lining membrane-spanning helix in the glutamate transporter GltT. Journal of Biological Chemistry 276:10775-10781.
Tolner, B., B. Poolman, and W. N. Konings. 1992. Characterization and functional expression in Escherichia coli of the sodium/proton/glutamate symport proteins of Bacillus stearothermophilus and Bacillus caldotenax. Molecular Microbiology 6:2845-2856.
Vagner, V., E. Dervyn, and S.D. Ehrlich. 1998. A vector for systematic gene inactivation in Bacillus subtilis. Microbiology 144:3097-3104.
Veening, J.-W., O. P. Kuipers, S. Brul, K. J. Hellingwerf, and R. Kort. 2006. Effects of Phosphorelay Perturbations on Architecture, Sporulation, and Spore Resistance in Biofilms of Bacillus subtilis. J. Bacteria 188:3099-3109.
Wang, W., and D. H. Bechhofer. 1996 Properties of a Bacillus subtilis polynucleotide phosphorylase deletion strain. Journal of Bacteriology 178:2375-2382.

1. A method of identifying an inhibitor of LtaS comprising:
(a) providing gram positive bacteria in which the bacteria comprise a mutation in the mbl gene or homologue thereof;
(b) culturing the bacteria of (a) in the presence of a test substance under conditions of low magnesium;
(c) monitoring the growth of the bacteria;
wherein growth or more rapid growth of the bacteria compared to growth in the absence of the test substance is indicative that the test substance is an inhibitor of LtaS.

2. A method according to claim 1, wherein the mutation in the mbl gene comprises deletion of part or all of the mbl gene.

3. A method according to claim 2, wherein the entire mbl gene is deleted.

4. A method according to claim 1, wherein the conditions of low magnesium comprise an amount of magnesium such that the bacteria grow at less than 10% of the rate of bacteria having the same mbl deletion when grown under conditions of 20 mM Mg²⁺.

5. A method according to claim 4, wherein the conditions of low magnesium comprise less than 1 mM Mg²⁺.

6. A method according to claim 4, wherein the bacteria are cultured in medium unsupplemented by additional Mg²⁺.

7. A method according to claim 1, wherein step (c) comprises monitoring the optical density of the culture to monitor for growth.

8. A method according to claim 7, wherein the method comprises growing an mbl mutant bacteria strain in the presence of high Mg²⁺, diluting into low Mg²⁺ medium and transferring to a sample tube, adding a test substance, and monitoring for bacterial growth by monitoring the optical density in the sample well.

9. A method according to 1, wherein the bacteria are cultured on an agar plate containing low Mg²⁺ medium, test substance is spotted onto the plate and bacterial growth is detected by visual inspection of the plate.

10. A method according to claim 9, wherein bacteria comprising the mbl mutant are cultured in high Mg²⁺ prior to dilution and spreading onto the agar plates.

11. A method according to claim 1, wherein the gram positive bacteria is a bacillus.

12. A method according to claim 11, wherein the bacillus is B. subtilis.

13. A method of producing an antibiotic comprising conducting the method according to any one of the preceding claims to identify an inhibitor of LtaS, and formulating the inhibitor in a pharmaceutical composition.


SEQUENCE LISTING (21 sequences)

SEQ ID NO: 1 - DNA, 2342 bp, Bacillus subtilis
  CDS (196)..(2142)
attcctttatttctagaaagataccttgttttacatttggtaatatcaaagcgaaacgttgattcgacggcgtttttcgccactttctccctcatacgattttcacttttctaatctgctgattcgtgttatattggatacgttcgttttttctatcgtttcgctcgaactggatcggaaaaaaggagtgtaacaatgaaaacatttataaaagaaagaggactggccttcttcttaattgcggtcgtcctgttatggatcaaaacgtatgtcggttatgtcctgaatttcaacttaggaatagacaacacgatacaaaaaatattgctttttgtgaatcctcttagctcaagcttgttctttcttggctttggactcttgttcaagaaaaaattacagcagacagccattatagtgattcattttttaatgtcttttttactgtacgccaacattgtgtactacagatttttcaatgattttattacaattccggtcattatgcaggctaaaacaaacggcggccaactcggtgacagcgcattttcgctgatgagaccgactgacgccttttactttatcgatacgatcatcctgatcatcttggcgatcaaagtaaacaagcctgccgaaacgtcaagcaaaaaatcgttccgaattatttttgcgtcttcaattcttgtgttcttgatcaacctggcagttgcggaatcagaccgtcctgaattgctgacaagatcattcgaccggaactatcttgtgaaatacttgggaacatacaatttcacgatttatgacgctgtacagaatatcaagtccaacagccagcgcgcgcttgccgattccagcgacgtaacggaagtagaaaactacatgaaagccaattacgatgtgccgaataacgtgtatttcggcaaagcggaaggaaaaaacgtcatttacgtttcacttgaatctttgcagtcatttatcatcgactataaaattgacggcaaagaagtgacaccattcttaaataaactggcacatgataacgaaacgttctactttgataactttttccaccaaacgggacaaggtaaaacatctgatgctgaatttatgatggaaaactctctgtacccgctggctcaaggttcagttttcgtaaacaaagcgcaaaacacgctgcaatccgttccggcgattctgaagtctaagaattacacatctgctactttccacgggaacacgcagacgttctggaaccgtaacgaaatgtacaaggcggaaggcattgataaattctttgattctgcttactatgacatgaacgaagaaaacacgaaaaactacggcatgaaagacaaaccgttcttcaaagaatcaatgccgctgctggaaagcctgccgcagccgttctatacgaagttcattaccctttccaaccacttcccattcggaatggatgagggggatacagacttcccggctggagactttggtgactctgtcgtcgataactatttccagtcagcccattaccttgatcagtccattgaacaattcttcaatgatctgaaaaaagacgggttatatgataaatcgattattgtgatgtacggagaccactacggcatctctgaaaaccacaataaagcgatggcgaaagtgcttggcaaggatgaaatcactgattacgacaacgcccagcttcaacgggtgccgctctttatccacgctgccggcgtgaagggcgagaaagttcataaatatgccggagacgttgatgtggctcctaccattctgcatctgctcggagtggatacgaaggactatctgatgtccggttctgatattttatcgaaagaacaccgtgaagtgattccgttccgaaacggagactttatttcaccgaagtacacgaaaatatccggtaagtattacgacacgaaaaccggaaaagaactcgatgaatccgaagtcgacaagtcagaagactcactcgtcaagaaggaacttgaaatgtccgataaaatcataaacggagacctgctgcggttctacgagccgaaaggttttaagaaggtgaatccttctgattatgattacacaaaacatgacgaagattcttccgaaacgtcaaaggataacgaagataaataagaaaaagcggagaggttgccctctccgcttttttatttgacagcagccctcaattcctcagctgcaaattccacattcgggccaataatgacttgaaccgattgcccgcccgatttgacaaccccttttgcgcctgctttctttagcagtgcttcatccaccaaagcggtatccttcacagtcagtcgcagccttgt

SEQ ID NO: 2 - protein, 649 aa, Bacillus subtilis
MKTFIKERGLAFFLIAVVLLWIKTYVGYVLNFNLGIDNTIQKILLFVNPLSSSLFFLGFGLLFKKKLQQTAIIVIHFLMSFLLYANIVYYRFFNDFITIPVIMQAKTNGGQLGDSAFSLMRPTDAFYFIDTIILIILAIKVNKPAETSSKKSFRIIFASSILVFLINLAVAESDRPELLTRSFDRNYLVKYLGTYNFTIYDAVQNIKSNSQRALADSSDVTEVENYMKANYDVPNNVYFGKAEGKNVIYVSLESLQSFIIDYKIDGKEVTPFLNKLAHDNETFYFDNFFHQTGQGKTSDAEFMMENSLYPLAQGSVFVNKAQNTLQSVPAILKSKNYTSATFHGNTQTFWNRNEMYKAEGIDKFFDSAYYDMNEENTKNYGMKDKPFFKESMPLLESLPQPFYTKFITLSNHFPFGMDEGDTDFPAGDFGDSVVDNYFQSAHYLDQSIEQFFNDLKKDGLYDKSIIVMYGDHYGISENHNKAMAKVLGKDEITDYDNAQLQRVPLFIHAAGVKGEKVHKYAGDVDVAPTILHLLGVDTKDYLMSGSDILSKEHREVIPFRNGDFISPKYTKISGKYYDTKTGKELDESEVDKSEDSLVKKELEMSDKIINGDLLRFYEPKGFKKVNPSDYDYTKHDEDSSETSKDNEDK

SEQ ID NO: 3 - DNA, 1399 bp, Bacillus subtilis
  CDS (201)..(1199)
aaattctcgaaggagagcctgttcagcaatcgtaatcacctggcattgccttcttgaaatcgttcataaaacatccgcaaaaatttgtaaagaacttattgtgcttccaactttttttctatattttatgataatatatataattagggcacaatgtggatatttactgtgaaacagattttcaaggaggatataaatagatgtttgcaagggatattggtattgacctcggtactgcaaatgtactgatccatgttaaaggtaaaggaattgttctgaatgaaccttccgttgttgcacttgataaaaacagcggcaaagtgctggcggttggcgaagaggcaagacgaatggttggacgtacacctgggaatattgttgcgattcgcccgctgaaagacggagttattgctgactttgaagtaacagaagcaatgctgaaacattttattaacaagctgaatgtaaaaggcctgttctcaaagccgcgcatgctcatttgctgcccgacgaatattacatccgttgagcaaaaagcaattaaagaagctgcagaaaaaagcggcgggaaacatgtgtaccttgaagaagaacctaaagttgccgctatcggcgcgggtatggaaatattccagccaagcggtaacatggttgtagacatcggaggcgggacgacggatatcgcggttatttcaatgggcgatattgtcacctcctcttctattaaaatggctggggacaagtttgacatggaaatcttaaattatatcaaacgcgagtacaagctgctgatcggcgaacgtactgcggaggatattaagattaaagtcgcaactgttttcccagacgcacgtcacgaggaaatttccattcgcggacgggacatggtttccggtcttccaagaacaattacagtaaacagtaaagaagttgaagaagcccttcgtgaatctgtcgctgttattgttcaggctgcaaaacaagtgctcgaaagaacaccgcctgaactttctgctgatattattgaccgcggcgttattattaccggcggaggcgcgctcttaaacggccttgaccagctgcttgctgaagagctgaaggtaccggtcctcgttgctgaaaatcctatggattgcgtagccatcggcacaggtgtcatgcttgataatatggacaagcttcctaaacgcaaactaagctgatttcacaaacctcattctgaaaaagaatgaggtttttttatgaaaaagccttcacgaaaagatgttaaatgacgataataggataaaatactgagtttttattatagaacgaacgttcctatatgacaactggaaaaaatgccatttttagaggtgggaaatttgttaaaaggattatatacagcaacatccgcaat

SEQ ID NO: 4 - protein, 333 aa, Bacillus subtilis
MFARDIGIDLGTANVLIHVKGKGIVLNEPSVVALDKNSGKVLAVGEEARRMVGRTPGNIVAIRPLKDGVIADFEVTEAMLKHFINKLNVKGLFSKPRMLICCPTNITSVEQKAIKEAAEKSGGKHVYLEEEPKVAAIGAGMEIFQPSGNMVVDIGGGTTDIAVISMGDIVTSSSIKMAGDKFDMEILNYIKREYKLLIGERTAEDIKIKVATVFPDARHEEISIRGRDMVSGLPRTITVNSKEVEEALRESVAVIVQAAKQVLERTPPELSADIIDRGVIITGGGALLNGLDQLLAEELKVPVLVAENPMDCVAIGTGVMLDNMDKLPKRKLS

SEQ ID NO: 5 - DNA, 23 bp, Artificial Sequence
  IPCR1 oligonucleotide
gcttgtaaattctatcataattg

SEQ ID NO: 6 - DNA, 21 bp, Artificial Sequence
  IPCR2 oligonucleotide
agggaatcatttgaaggttgg

SEQ ID NO: 7 - DNA, 21 bp, Artificial Sequence
  IPCR3 oligonucleotide
gcatttaatactagcgacgcc

SEQ ID NO: 8 - DNA, 33 bp, Artificial Sequence
  mbl-A oligonucleotide
gctcactctagaccgaggtcaataccaatatcc

SEQ ID NO: 9 - DNA, 18 bp, Artificial Sequence
  mbl-B oligonucleotide
gtgatgaagcgtcctatg

SEQ ID NO: 10 - DNA, 33 bp, Artificial Sequence
  mbl-C oligonucleotide
ctgagcgaattccgcaaactaagctgatttcac

SEQ ID NO: 11 - DNA, 19 bp, Artificial Sequence
  mbl-D oligonucleotide
cctatatggcctggaagac

SEQ ID NO: 12 - DNA, 30 bp, Artificial Sequence
  mbl-fw oligonucleotide
ctcgaggatccacctggcattgccttcttg

SEQ ID NO: 13 - DNA, 32 bp, Artificial Sequence
  mbl-rev oligonucleotide
catactgaattccatgacacctgtgcccgatg

SEQ ID NO: 14 - DNA, 30 bp, Artificial Sequence
  yflE-A1 oligonucleotide
ctagcagcatgcgttcgagcgaaacgatag

SEQ ID NO: 15 - DNA, 30 bp, Artificial Sequence
  yflE_A2 oligonucleotide
gtacggtctagagttcgagcgaaacgatag

SEQ ID NO: 16 - DNA, 19 bp, Artificial Sequence
  yflE-B oligonucleotide
catcgtgattccggcactc

SEQ ID NO: 17 - DNA, 29 bp, Artificial Sequence
  yflE-C1 oligonucleotide
catctaggtaccgagaggttgccctctcc

SEQ ID NO: 18 - DNA, 29 bp, Artificial Sequence
  yflE_C2 oligonucleotide
ctagctgaattcgagaggttgccctctcc

SEQ ID NO: 19 - DNA, 19 bp, Artificial Sequence
  yflE-D oligonucleotide
ctgccgtaatgcatgtcag

SEQ ID NO: 20 - DNA, 30 bp, Artificial Sequence
  yflEfw oligonucleotide
gacagtggatcccactttctccctcatacg

SEQ ID NO: 21 - DNA, 30 bp, Artificial Sequence
  yflErev oligonucleotide
catccagaattcgcagctgaggaattgagg